(19) United States
(12) Patent Application Publication
(10) Pub. No.: US 2004/0267212 A1
(43) Pub. Date: Dec. 30, 2004

(54) Diverging tip aortic cannula

(76) Inventor: Kenneth R. Jonkman, Crestline, CA (US)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/735,261

(22) Filed: Dec. 12, 2003

Related U.S. Application Data
(60) Provisional application No. 60/482,923, filed on Jun. 27, 2003.

(51) Int. Cl. A61M 31/00

(52) U.S. Cl. 604/275

(57) ABSTRACT

Dispersion nozzles (10) are disclosed which are to be deployed on the terminus of an aortic cannula (12). The dispersion nozzles (10) each include a collar (16) with a passage therethrough. Curved vanes (18) extend across the flow path on the distal end of the collar (16). An arch (20) bisects the vanes 18 at their outer edges. The vanes (18) and the arch (20) direct flow into a substantially hemispherical flow profile to increases the cross-sectional area of the flow stream from the cannula (12).

### REFERENCE TO RELATED APPLICATIONS

This application claims priority from U.S. Provisional Application No. 60/482,923, filed Jun. 27, 2003 in the name of Kenneth R. Jonkman, the disclosure of which is incorporated herein by reference.

### BACKGROUND OF THE INVENTION

The field of the present invention is aortic cannula.

The following describes an aortic cannula having an outlet which reduces the flow velocity of fluid, particularly blood, as it exits the cannula to avoid dislodging of material from the aorta wall. Other mechanisms for reducing flow velocity at the tip of an aortic cannula are disclosed in U.S. Pat. Nos. 5,354,288; 5,643,226; 5,685,865; 5,976,114; and 6,059,760. The disclosures of these five patents are incorporated herein by reference.

### SUMMARY OF THE INVENTION

The present invention is directed to an aortic cannula having a dispersion nozzle at the terminus thereof. The dispersion nozzle includes curved vanes to direct flow into a substantial hemispherical flow profile. The nozzle greatly increases the cross-sectional area of the flow stream, reducing the velocity of flow from that at the terminus of the aortic cannula.

Accordingly, it is an object of the present invention to provide an improved aortic cannula. Other and further objects and advantages will become apparent from the following.

### BRIEF DESCRIPTION OF THE DRAWING

FIG. 1 is a side view of a dispersion nozzle.

FIG. 2 is a perspective view of the dispersion nozzle on a cannula with the cannula partially broken away for clarity and only a short length illustrated.

FIG. 3 is a side view of a second embodiment of a dispersion nozzle.

FIG. 4 is a perspective view of the dispersion nozzle of FIG. 3.

FIG. 5 is a second perspective view of the dispersion nozzle of FIG. 3.

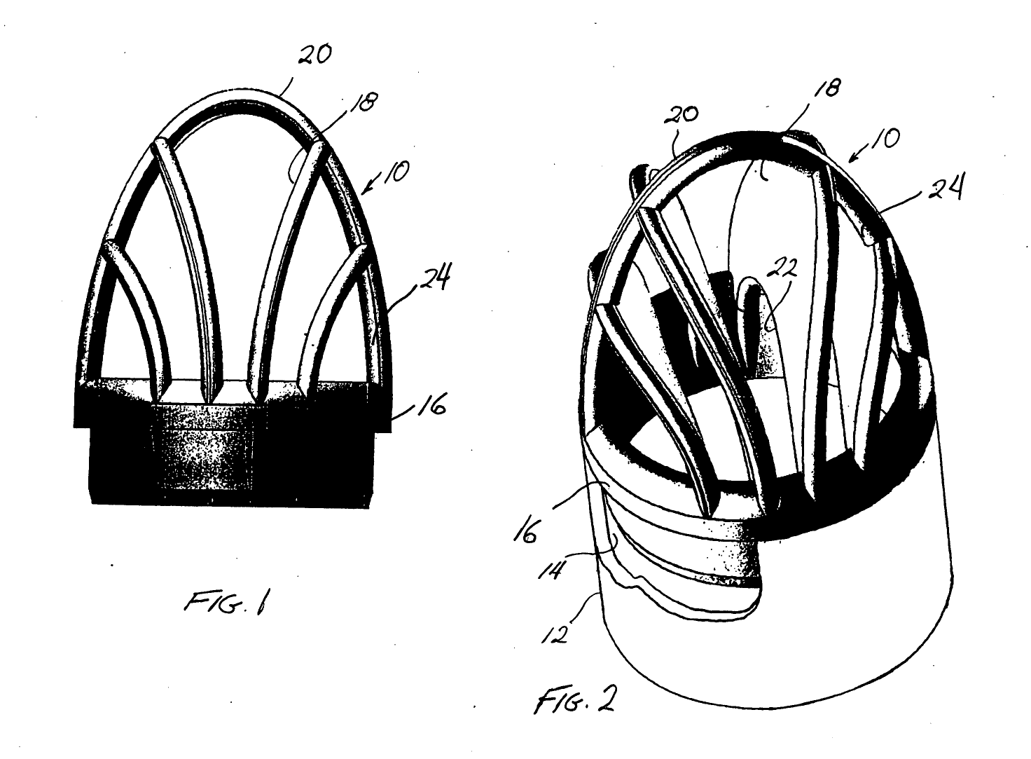

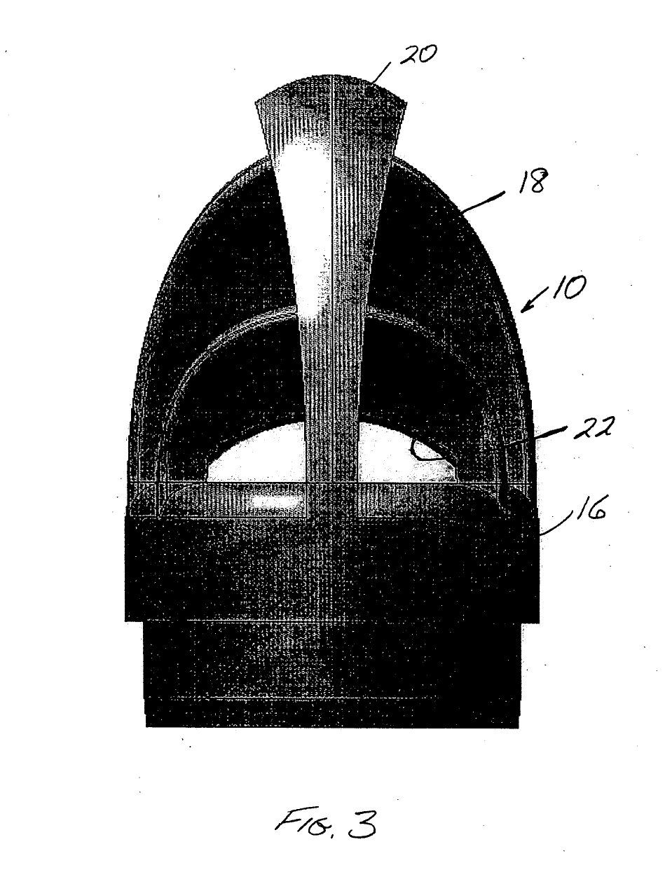

### DETAILED DESCRIPTION OF THE PREFERRED EMBODIMENT

Turning in detail to the drawing, two dispersion nozzles, generally identified as 10, are disclosed which are to be deployed on the terminus of an aortic cannula 12 of know construction with at least one lumen 14. The dispersion nozzles 10 each includes a collar 16 with a passage therethrough. The collar may be attached by adhesive or embedded into the laminated structure of the cannula 12. Vanes 18 extend across the flow path on the distal end of the collars 16. Arches 20 bisect the vanes 18 at their outer edges on each nozzle. Each entire dispersion nozzle 10 is most conveniently of single piece molded medical grade polymer.

The vanes 18 are each formed with an arched inner edge 22 of significant width. As the arches 22 are higher for the middle vanes 12, a domed inner cavity is defined within the collar 16. In effect, this feature gives increased entrance area to the nozzle passages between vanes 18. Additionally, the stream from the end of the lumen will be spread as the outer periphery of that stream first encounters resistance and diversion before the rapidly moving center flow. The blunt leading edges at the arches 22 of the vanes 18 operate to disrupt flow to effect a greater dispersive effect as flow passes through and from the nozzle passages.

The vanes 18 also diverge from one another as illustrated to generate multiple expanding flow paths. With this configuration and the rate of flow, the fluid flow is unlikely to leave the boundary layers defined by each of the vanes 18. As the stream from the end of the lumen 14 within the aortic cannula 12 is forced into an increased area of flow, flow velocity is reduced.

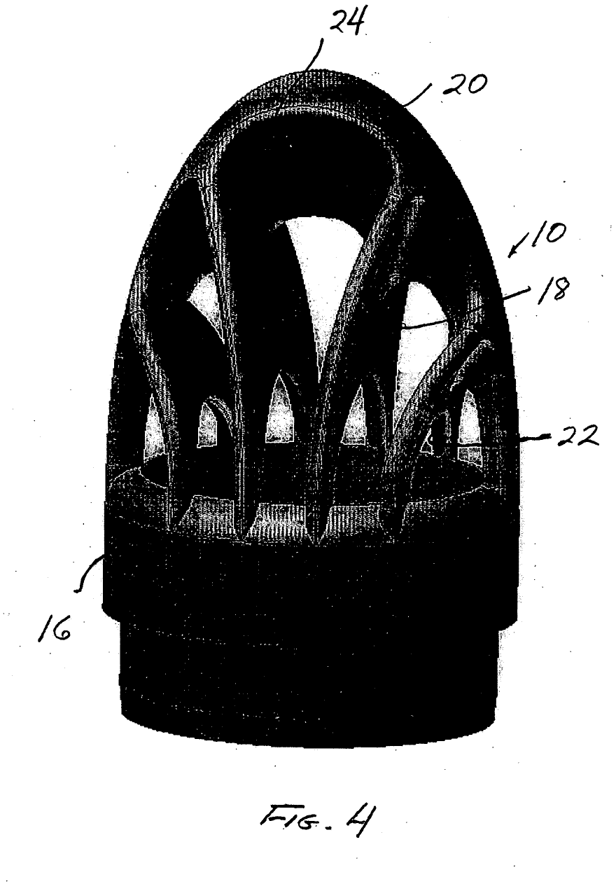

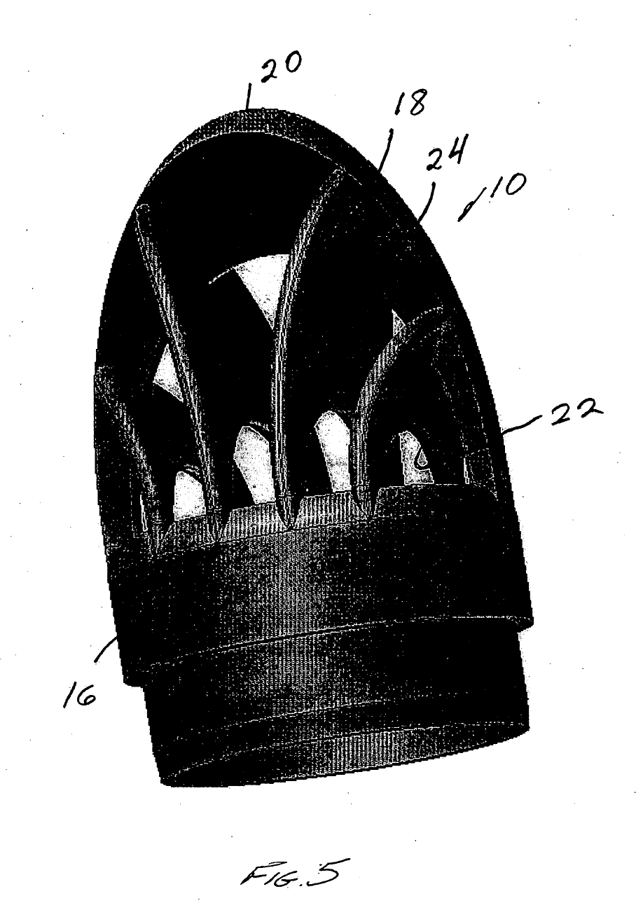

The arches 20 provide strength and protection to the vanes 18 and insure a smoother leading exterior to avoid engagement with the body on each nozzle 10. The arches 20 may be configured to operate to further disperse the flow in directions laterally parallel to the vanes 18 by including side surfaces 24 diverging one from another away from the terminus of the cannula. This divergence is most pronounced in the second embodiment of FIGS. 3, 4 and 5.

Thus, an improved aortic cannula has been described. While embodiments and applications of this invention have been shown and described, it would be apparent to those skilled in the art that many more modifications are possible without departing from the inventive concepts herein. The invention, therefore is not to be restricted except in the spirit of the appended claims.

What is claimed is:

1. An aortic cannula comprising
a cannula including a terminus and a lumen therethrough;
a dispersion nozzle at the terminus of the cannula including a plurality of curved vanes to direct flow into a substantially hemispherical flow profile to increase the cross-sectional area of the flow stream.

2. The aortic cannula of claim 1, the dispersion nozzle further including a collar having a passage therethrough, the vanes being attached to the collar across the passage therethrough, the curved vanes having blunt leading edges defining a domed inner cavity within the collar.

3. The aortic cannula of claim 1, the dispersion nozzle further including an arch bisecting the vanes at the outer edges of the vanes.

4. The aortic cannula of claim 3, the arch having side surfaces diverging one from another away from the terminus of the cannula.

5. The aortic cannula of claim 1, where the curved vanes are divergent from each other away from the terminus of the cannula.

6. An aortic cannula comprising
a cannula including a terminus and a lumen therethrough;
a dispersion nozzle at the terminus of the cannula including a plurality of curved vanes to direct flow into a substantially hemispherical flow profile to increase the cross-sectional area of the flow stream and a collar having a passage therethrough, the curved vanes being attached to the collar across the passage therethrough, having blunt leading edges defining a domed inner cavity within the collar and being divergent from each other away from the terminus of the cannula.

7. The aortic cannula of claim 6, the dispersion nozzle further including an arch bisecting the vanes at the outer edges of the vanes.

8. An aortic cannula comprising
a cannula including a terminus and a lumen therethrough;
a dispersion nozzle at the terminus of the cannula including a plurality of curved vanes to direct flow into a substantially hemispherical flow profile to increase the cross-sectional area of the flow stream, a collar having a passage therethrough and an arch bisecting the vanes at the outer edges of the vanes, the curved vanes being attached to the collar across the passage therethrough, having blunt leading edges defining a domed inner cavity within the collar and being divergent from each other away from the terminus of the cannula, the arch having side surfaces diverging one from another away from the terminus of the cannula.
